(19) United States
(12) Patent Application Publication
(10) Pub. No.: US 2025/0268505 A1
(43) Pub. Date: Aug. 28, 2025

(54) HYBRID-TYPE NEURAL ELECTRODE

(71) Applicant: DAEGU GYEONGBUK INSTITUTE OF SCIENCE AND TECHNOLOGY, Daegu (KR)

(72) Inventors: So Hee KIM, Daegu (KR); Jae Won JANG, Daegu (KR)

(21) Appl. No.: 18/857,505

(22) PCT Filed: Jun. 8, 2023
(86) PCT No.: PCT/KR2023/007800

§ 371 Date: Oct. 17, 2024

(30) Foreign Application Priority Data
Jul. 27, 2022 (KR) 10-2022-0093530

(51) Int. Cl. A61B 5/294; A61B 5/388

(52) U.S. Cl. CPC A61B 5/294; A61B 5/388

(57) ABSTRACT

A hybrid-type neural electrode is disclosed. The hybrid-type neural electrode comprises: a first layer including a bottom surface on which a planar electrode is arranged and a plurality of holes formed to penetrate vertically; and a second layer including a bottom surface on which a measurement device for measuring a neural signal is arranged while facing the top surface of the first layer.

### TECHNICAL FIELD

The present disclosure relates to a hybrid type neural electrode, and more particularly, to a hybrid type neural electrode having a structure improved such that various types of electrodes are integrated.

### BACKGROUND ART

Neural electrodes in the related art measure only electrocorticogram (ECOG) through non-invasive electrodes, or measure action potentials in single neurons or local field potentials from small groups of neurons through invasive electrodes within the brain.

In the case of the related art neural electrode in which an invasive electrode and a non-invasive electrode within the brain are formed integrally with each other, the length of the invasive electrode is limited due to processing limitations, making it difficult to acquire biological signals from various areas.

### DESCRIPTION OF EMBODIMENTS

### Technical problem

The present disclosure has been invented to solve the above-mentioned problems, and an aspect of the present disclosure is to provide a hybrid type neural electrode having a structure improved such that various types of electrodes are integrated.

### Solution to Problem

According to an aspect of the present disclosure, there is provided a hybrid type neural electrode that may include a first layer that includes a lower surface on which a planar electrode is arranged, and a plurality of holes vertically formed therethrough, and a second layer that includes a lower surface which faces an upper surface of the first layer and on which a measuring device for measuring neural signals is arranged.

### BRIEF DESCRIPTION OF DRAWINGS

FIG. 1 is a perspective view illustrating a hybrid type neural electrode according to one embodiment of the present disclosure.

FIG. 2 is a diagram illustrating a structure in which a plurality of invasive electrodes are connected to an upper layer of the hybrid type neural electrode.

FIG. 3 is a diagram illustrating a structure in which a single invasive electrode is connected to the upper layer of the hybrid type neural electrode.

FIG. 4 is a diagram illustrating a structure in which an optical stimulation device is connected to the upper layer of the hybrid type neural electrode.

### BEST MODE

According to an aspect of the present disclosure, there is provided a hybrid type neural electrode that may include a first layer that includes a lower surface on which a planar electrode is arranged, and a plurality of holes vertically formed therethrough, and a second layer that includes a lower surface which faces an upper surface of the first layer and on which a measuring device for measuring neural signals is arranged.

The measuring device may include an invasive electrode that has an upper end electrically connected to the lower surface of the second layer and penetrates each of the plurality of holes of the first layer.

The invasive electrode may include a connection part that has a front surface electrically connected to the second layer, an insertion part that extends downward from the connection part to be inserted into a body, and a light source part that is arranged on the insertion part and irradiates light for stimulating neurons.

The measuring device may include an optical stimulation device that irradiates light downward from the lower surface of the second layer toward a corresponding hole of each of the plurality of holes of the first layer, the light stimulating the neurons.

The planar electrode may be arranged on a front end of the first layer, the measuring device may be arranged on a front end of the second layer, rear ends of the first and second layers may be integrally connected to each other, and the front ends of the first and second layers may be formed separately from each other.

The hybrid type neural electrode may further include a connection part that connects the rear ends of the first and second layers with each other, and the connection part may include an output unit that is electrically connected to an external device to output a neural signal to the external device, and a transmission unit that electrically connects the first and second layers with the output unit to transmit neural signals.

### MODE FOR INVENTION

It should be understood that the embodiments disclosed herein are illustrative only for better understanding of the present disclosure, and that the present disclosure may be modified in various ways. However, in describing the present disclosure, if it is determined that a detailed description of a known function or component may unnecessarily obscure the gist of the present disclosure, the detailed description and illustration will be omitted. In addition, for ease understanding of the present disclosure, the accompanying drawings are not drawn to real scale, but the dimensions of some components may be exaggerated.

The terms used in the disclosure and claims are general terms selected in consideration of functions of the present disclosure. However, these terms may vary depending on intentions of one of ordinary skill in the art, legal or technical interpretation, the advent of new technologies, and the like. Also, some of the terms used herein may be arbitrarily chosen by the present applicant. These terms may be interpreted as meaning as defined herein, and, unless otherwise specifically defined, may be interpreted based on the overall content of the present disclosure and common technical knowledge in the art.

As used herein, the terms “have,” “may have,” “include,” or “may include” a feature (e.g., a number, function, operation, or a component such as a part) indicate the existence of the feature and do not exclude the existence of other features.

In addition, in the specification, components necessary for description of each embodiment of the disclosure are described, and thus are not necessarily limited thereto. Therefore, some components may be changed or omitted, and other components may be added. In addition, the components may be disposed to be distributed in different independent apparatuses.

Further, hereinafter, embodiments of the disclosure will be described in detail with reference to the accompanying drawings and the contents described in the accompanying drawings, but the disclosure is not limited to or limited by the embodiments.

Hereinafter, the present disclosure will be described in more detail with reference to the attached drawings.

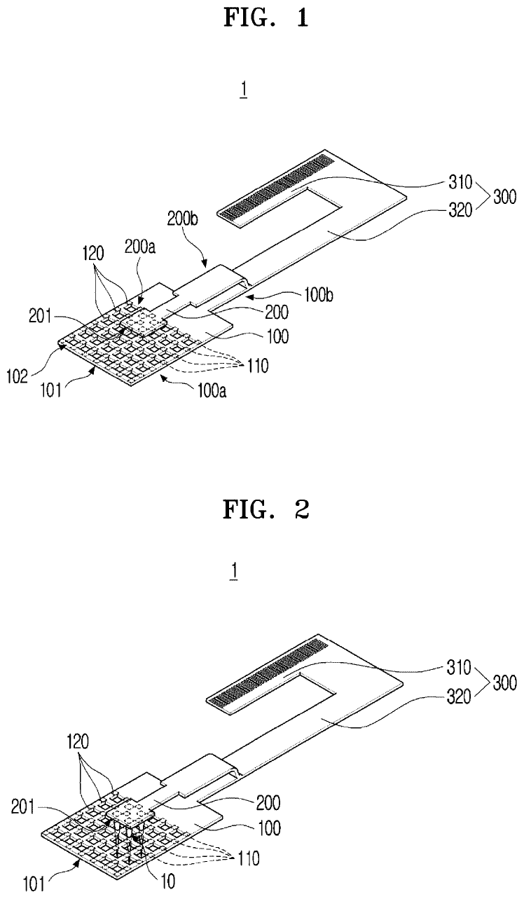

FIG. 1 is a perspective view illustrating a hybrid type neural electrode according to one embodiment of the present disclosure.

Referring to FIG. 1, a hybrid type neural electrode 1 according to one embodiment of the present disclosure may include a first layer 100 and a second layer 200.

The first layer 100 may include a lower surface 101 on which planar electrodes 110 are disposed and a plurality of holes 120 formed therethrough vertically. The first layer 100 may have the shape of a grid-structured plate formed by the plurality of holes 120. For example, the plurality of holes 120 of the first layer 100 may be arranged in a grid shape, but may not be limited to the arrangement, and may be arranged in various structures for the first layer 100 to have a grid structure.

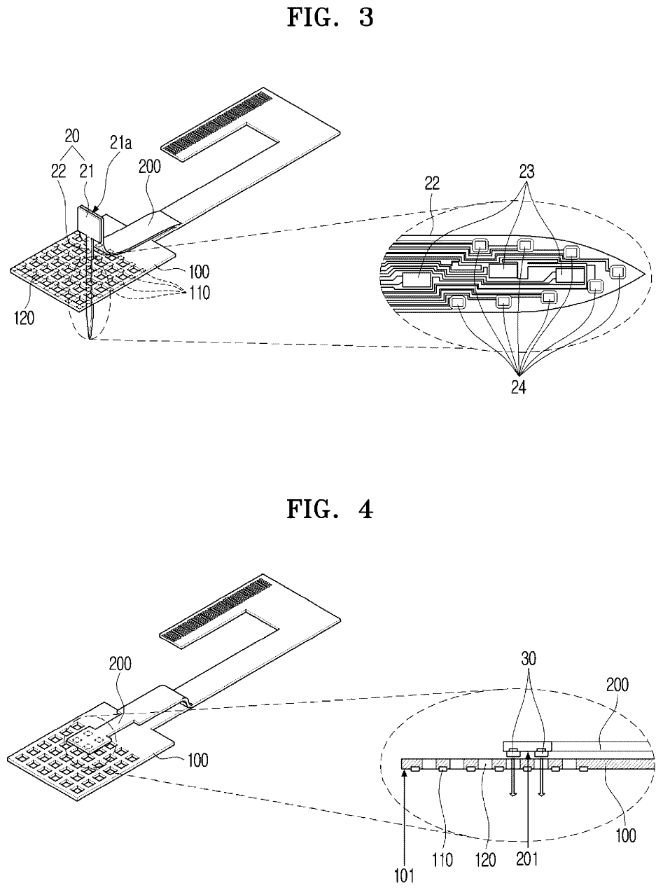

The second layer 200 may include a lower surface 201 which faces an upper surface 102 of the first layer 100 and on which a measuring device (not illustrated) for measuring neural signals is disposed. The measuring device may be any one of a plurality of invasive electrodes 10 in FIG. 2, a single invasive electrode 20 in FIG. 3, and an optical stimulation device 30 in FIG. 4, but the type is not limited thereto.

The first and second layers 100 and 200 may be made of a flexible material, for example, an elastomer, so that an electrode arrangement can be easily in close contact with a specific area of a biological tissue. Accordingly, the first and second layers 100 and 200 can be curved to fit the outer shape of the cerebral cortex.

The planar electrode 110 of the first layer 100 may be disposed on a front end 100a of the first layer 100. The measuring device for measuring neural signals may be disposed on a front end 200a of the second layer 200. The planar electrode 110 may be made of a metal material. The planar electrode 110 has a planar shape so as to be in contact with a surface of a biological tissue.

In the hybrid type neural electrode 1 having the two upper and lower layers, the planar electrode 110 of the first layer 100 and the measuring device of the second layer 200 may measure neural signals in the brain in various forms.

The second layer 200 is a connecting portion with an invasive electrode or another electrode, and is connectable to a device for various types of electrical stimulations or measurements. The first layer 100 enables the acquisition of neural signals in the form of ECoG through close contact with a brain surface, making it possible to measure signals in various areas.

A rear end 100b of the first layer 100 and a rear end 200b of the second layer 200 may be integrally connected to each other, and the front end 100a of the first layer 100 and the front end 200a of the second layer 200 may be formed separately from each other.

The hybrid type neural electrode 1 according to one embodiment of the present disclosure may further include a connection part 300. The connection part 300 may connect the rear end 100b of the first layer 100 and the rear end 200b of the second layer 200.

The connection part 300 may include an output unit 310 and a transmission unit 320. The output unit 310 may be electrically connected to an external device to output a neural signal to the external device. The transmission unit 320 may transmit neural signals by electrically connecting the first and second layers 100 and 200 and the output unit 310.

FIG. 2 is a diagram illustrating a structure in which a plurality of invasive electrodes are connected to an upper layer of the hybrid type neural electrode.

Referring to FIG. 2, a measuring device according to one embodiment of the present disclosure may include an invasive electrode 10. An electrode portion made of metal may be exposed at a distal end of the invasive electrode 10. For example, the invasive electrode 10 may have a pointed end so as to directly invade a biological tissue, but the shape of the invasive electrode 10 is not limited to this.

The invasive electrode 10 may include an upper end electrically connected to the lower surface 201 of the second layer 200. The invasive electrode 10 may penetrate the hole 120 of the first layer 100.

The invasive electrode 10 and the planar electrode 110 may apply electrical stimulation at different depths to the same point on the biological tissue or detect biological signals (e.g., ECoG signals or action potentials) according to the applied stimulation.

The invasive electrode 10 and the planar electrode 110 may each receive an electrical signal from outside and accordingly apply appropriate stimulation to a specific area of the biological tissue. In addition, the invasive electrode 10 and the planar electrode 110 may also serve to detect biological signals that appear in the specific area of the biological tissue due to the applied stimulation.

Accordingly, the planar electrode 110 and the invasive electrode 10 can independently measure neural signals without interference with each other. That is, in the hybrid type neural electrode 1 according to one embodiment of the present disclosure, two different types of devices can easily measure neural signals in a compact structure without interference with each other.

At least one of the first layer 100, the second layer 200, and the connection part 300 may include a feeding part (not illustrated) that applies an electrical signal to the invasive electrode 10 and the planar electrode 110 or detects a biological signal from the invasive electrode 10 and the planar electrode 110, and the invasive electrode 10 and the planar electrode 110 may be electrically connected to the feeding part.

FIG. 3 is a diagram illustrating a structure in which a single invasive electrode is connected to the upper layer of the hybrid type neural electrode.

Referring to FIG. 3, the invasive electrode 20 according to one embodiment of the present disclosure may include a connection part 21, an insertion part 22, and a light source part 23.

The connection part 21 may include a front surface 21a electrically connected to the second layer 200. The insertion part 22 may be formed to extend downward from the connection part 21 to be inserted into a body. The light source part 23 may be arranged on the insertion part 22 to irradiate light for stimulating neurons. The light source part 23 may be formed of at least one of optical fiber, LED, and OLED, but the type is not limited thereto.

Light irradiated from the light source part 23 may stimulate specific neurons. The light irradiated from the light source part 23 may cause neurons in a desired neural area to respond to light of a specific wavelength. Genetically modified neurons respond when exposed to light in a specific wavelength range and send out neural signals. This can allow more selective stimulation for single neurons, compared to existing electrical neural stimulation.

In addition to the light source part 23, a plurality of electrodes 24 may be arranged on the insertion part 22. The plurality of electrodes 24 may measure electrical signals, and the measured signals may be transmitted through a circuit disposed in the insertion part 22. The electrodes 24 may detect biological signals according to stimulation applied from the light source part 23.

In addition, since the plurality of electrodes 24 are arranged along a longitudinal direction of the insertion part 22, the plurality of electrodes 24 may measure neural signals at various positions along a depth direction of a biological tissue.

The light source part 23 may be provided in plurality which are arranged along the longitudinal direction of the insertion part 22. Accordingly, the invasive electrode 20 according to one embodiment of the present disclosure can stimulate neurons at various points in the depth direction, enabling more precise measurement and analysis of neural signals.

Also, the planar electrode 110 and the invasive electrode 20 can independently measure neural signals without interference with each other. That is, in the hybrid type neural electrode 1 according to one embodiment of the present disclosure, two different types of devices can easily measure neural signals in a compact structure without interference with each other.

FIG. 4 is a diagram illustrating a structure in which an optical stimulation device is connected to the upper layer of the hybrid type neural electrode.

Referring to FIG. 4, a measuring device according to one embodiment of the present disclosure may include an optical stimulation device 30. The optical stimulation device 30 may be formed of at least one of optical fiber, LED, and OLED, but the type is not limited thereto.

The optical stimulation device 30 may stimulate specific neurons, similar to the light source part (23 in FIG. 3). Light irradiated from the optical stimulation device 30 may cause neurons in a desired neural area to respond to light of a specific wavelength. Genetically modified nerve cells respond when exposed to light in a specific wavelength range and send out neural signals. This can allow more selective stimulation for single neurons, compared to existing electrical neural stimulation.

The optical stimulation device 30 may be disposed to irradiate light, which stimulates neurons, downward from the lower surface 201 of the second layer 200 toward the hole 120 of the first layer 100. The optical stimulation device 30 may be provided in plurality which are arranged at positions corresponding to the holes 120 of the first layer 100. The plurality of optical stimulation devices 30 may irradiate light of different wavelengths. For example, the plurality of optical stimulation devices 30 may be arranged in a grid form, but are not limited thereto, and may be arranged in various structures corresponding to the holes 120. Accordingly, light irradiated from the optical stimulation device 30 can easily proceed downward without interference with the first layer 100.

The light emitted from the optical stimulation device 30 can easily stimulate neurons without interference with the planar electrode 110. Additionally, the planar electrode 110 can easily measure neural signals without interference with the light irradiated from the optical stimulation device 30. That is, in the hybrid type neural electrode 1 according to one embodiment of the present disclosure, two different types of devices can easily measure neural signals in a compact structure without interference with each other.

So far, the preferred embodiment of the present disclosure has been shown and described, but the present disclosure is not limited to the specific embodiment described above, and can be changed and modified in various ways by anyone skilled in the art to which the present disclosure pertains without departing from the gist of the present disclosure as claimed in the claims and the changes and modifications fall within the scope of the claims.

### INDUSTRIAL APPLICABILITY

According to an embodiment of the present disclosure, a hybrid type neural electrode is provided. Additionally, embodiments of the present disclosure can be applied to, for example, commercially available neural electrodes.

1. A hybrid type neural electrode comprising:
a first layer that comprises a lower surface on which a planar electrode is arranged, and a plurality of holes vertically formed therethrough; and
a second layer that comprises a lower surface which faces an upper surface of the first layer and on which a measuring device for measuring neural signals is arranged.

2. The hybrid type neural electrode of claim 1,
wherein the measuring device comprises
an invasive electrode that has an upper end electrically connected to the lower surface of the second layer and penetrates each of the plurality of holes of the first layer.

3. The hybrid type neural electrode of claim 2,
wherein the invasive electrode comprises:
a connection part that has a front surface electrically connected to the second layer;
an insertion part that extends downward from the connection part to be inserted into a body; and
a light source part that is arranged on the insertion part and irradiates light for stimulating neurons.

4. The hybrid type neural electrode of claim 1,
wherein the measuring device comprises
an optical stimulation device that irradiates light downward from the lower surface of the second layer toward a corresponding hole of the plurality of holes of the first layer, the light stimulating the neurons.

5. The hybrid type neural electrode of claim 1,
wherein the planar electrode is arranged on a front end of the first layer,
the measuring device is arranged on a front end of the second layer,
rear ends of the first and second layers are integrally connected to each other, and
the front ends of the first and second layers are formed separately from each other.

6. The hybrid type neural electrode of claim 5, further comprising
a connection part that connects the rear ends of the first and second layers with each other, and
wherein the connection part comprises:
an output unit that is electrically connected to an external device to output a neural signal to the external device; and
a transmission unit that electrically connects the first and second layers with the output unit to transmit a neural signal.
